(19) United States
(12) Patent Application Publication
(10) Pub. No.: US 2009/0093682 A1
(43) Pub. Date: Apr. 9, 2009

(54) SURGICAL PORTAL WITH FOAM AND FABRIC COMPOSITE SEAL ASSEMBLY

(75) Inventors: Steven L. Izzo, Naugatuck, CT (US); Michael J. Bettuchi, Middletown, CT (US); Frank R. Schiretz, JR., Middletown, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/244,138

(22) Filed: Oct. 2, 2008

Related U.S. Application Data
(60) Provisional application No. 60/997,845, filed on Oct. 5, 2007.

(51) Int. Cl. A61B 17/34

(52) U.S. Cl. 600/201

(57) ABSTRACT

A surgical portal assembly includes a portal adapted to provide access to underlying tissue and having a longitudinal opening extending along a longitudinal axis of the portal and a seal. The seal includes internal surfaces having a passage for reception and passage of a surgical object in substantial sealed relation therewith and defines a seal axis. The seal includes a foam segment comprising a foam material and a fabric segment comprising a fabric material and being mounted relative to the foam segment. The fabric segment may be disposed adjacent one of the proximal and distal surfaces of the foam segment. The fabric segment may include a fabric layer which is in juxtaposed relation with the one of the proximal and distal surfaces of the foam segment. The fabric layer may include slots therethrough to facilitate passage of the surgical object through the seal. The slots may be arranged to extend radially outwardly relative to the seal axis.

### CROSS REFERENCE TO RELATED APPLICATION

The present application claims the benefit of and priority to U.S. Provisional Application Ser. No. 60/997,845 filed on Oct. 5, 2007, the entire contents of which are incorporated herein by reference.

### BACKGROUND

1. Field of the Disclosure

The present disclosure relates to surgical devices and, more particularly, relates to a surgical portal apparatus incorporating a seal assembly adapted for use during a minimally invasive, e.g., a laparoscopic, surgical procedure.

2. Description of the Related Art

Minimally invasive surgical procedures including both endoscopic and laparoscopic procedures permit surgery to be performed on organs, tissues and vessels far removed from an opening within the tissue. Laparoscopic and endoscopic procedures generally require that any instrumentation inserted into the body be sealed, e.g., provisions may be made to ensure that gases do not enter or exit the body through the incision as, for example, in surgical procedures in which the surgical region is insufflated. These procedures typically employ surgical instruments which are introduced into the body through a cannula. The cannula has a seal assembly associated therewith. The seal assembly is intended to form a substantially fluid tight seal about the instrument to preserve the integrity of the established pneumoperitoneum.

### SUMMARY

The present disclosure is directed to further improvements in seal assemblies for use with portal apparatii during a surgical procedure. In accordance with one embodiment, a surgical portal assembly includes a portal adapted to provide access to underlying tissue and having a longitudinal opening extending along a longitudinal axis of the portal and a seal. The seal includes internal surfaces having a passage for reception and passage of a surgical object in substantial sealed relation therewith and defines a seal axis. The seal includes a foam segment comprising a foam material and a fabric segment comprising a fabric material and being mounted relative to the foam segment. The fabric segment may be disposed adjacent one of the proximal and distal surfaces of the foam segment. The fabric segment may include a fabric layer which is in juxtaposed relation with the one of the proximal and distal surfaces of the foam segment. The fabric layer may include slots therethrough to facilitate passage of the surgical object through the seal. The slots may be arranged to extend radially outwardly relative to the seal axis.

The seal may have first and second fabric layers mounted in juxtaposed relation with respective proximal and distal surfaces of the foam segment. Alternatively, the seal may further include an elastomeric segment comprising an elastomeric material having less elasticity than the foam material of the foam segment. The elastomeric segment is mounted in juxtaposed relation to the fabric layer. The seal may include, from proximal to distal, the elastomeric segment, the fabric layer and the foam segment. A second layer of fabric may be mounted adjacent the distal surface of the foam segment and a third layer of fabric may be mounted adjacent a proximal surface of the elastomeric segment.

The seal may include an outer seal area and an inner seal area. The inner seal area generally tapers in a distal direction to define a general funnel configuration or a general sloped configuration which is adapted to facilitate insertion of the surgical object and minimize potential of inversion of the inner seal area upon withdrawal of the surgical object.

The portal may include a portal housing and a portal sleeve extending from the portal housing. In this embodiment, the seal may include a substantially annular retention member which is received within a corresponding annular recess of the portal housing to assist in mounting the seal within the portal housing.

### BRIEF DESCRIPTION OF THE DRAWINGS

Preferred embodiments of the present disclosure will be better appreciated by reference to the drawings wherein:

FIG. 1 is a perspective views of a portal system in the form of a cannula assembly and a seal assembly in accordance with the principles of the present disclosure;

FIG. 2 is a side plan view of the portal system of FIG. 1;

FIG. 3 is a side cross-sectional view of the portal system;

FIG. 4 is an enlarged view of the area of detail depicted in FIG. 3;

FIG. 5 is a perspective view illustrating the seal assembly detached from the cannula assembly;

FIG. 6 is a perspective view with parts separated of the seal assembly illustrating the seal housing, the object seal and the locking ring;

FIGS. 7A, 7B and 7C are respective plan, perspective and side-cross-sectional views of the object seal;

FIG. 7D is a perspective view with parts illustrating the components of the object seal;

FIGS. 8A, 8B and 8C are respective front, side and rear plan views of the locking ring;

FIGS. 9A-9E are views illustrating a sequence of assembling the seal components of the seal assembly; and

FIG. 10 is a flow chart illustrating a method for performing a surgical task with the portal system of FIG. 1.

### DETAILED DESCRIPTION

The portal system of the present disclosure incorporates a seal assembly which, either alone or in combination with a seal internal to a cannula assembly, provides a substantial seal between a body cavity of a patient and the outside atmosphere before, during and after insertion of an object through the cannula assembly. Moreover, the seal assembly is capable of accommodating objects of varying diameters, e.g., instruments from about 4.5 mm to about 15 mm, by providing a gas tight seal with each instrument when inserted. The flexibility of the seal assembly greatly facilitates endoscopic surgery where a variety of instruments having differing diameters are often needed during a single surgical procedure.

The seal assembly contemplates the introduction and manipulation of various types of instrumentation adapted for insertion through a trocar and/or cannula assembly while maintaining a fluid tight interface about the instrumentation to prevent gas and/or fluid leakage from the established pneumoperitoneum so as to preserve the atmospheric integrity of a surgical procedure. Specifically, the seal assembly accommodates angular manipulation of the surgical instrument relative to the seal axis. This feature of the seal assembly desirably minimizes the entry and exit of gases and/or fluids to/from the body cavity. Examples of instrumentation include clip appliers, graspers, dissectors, retractors, staplers, laser probes, photographic devices, endoscopes and laparoscopes, tubes, and the like. Such instruments will be collectively referred to herein as “instruments or instrumentation”.

The seal assembly may be a component of a portal system adapted to provide access to an underlying site. The seal assembly may be readily incorporated into a portal, such as a conventional trocar device or cannula, and provides the device with sealing capability about an inserted instrument.

The seal assembly may also be adapted to receive and form a seal about a physician's arm or hand during a hand-assisted laparoscopic procedure. In this application, the seal assembly is a component of an access member which is introduced within the body to provide access to underlying tissue in, e.g., the abdominal cavity.

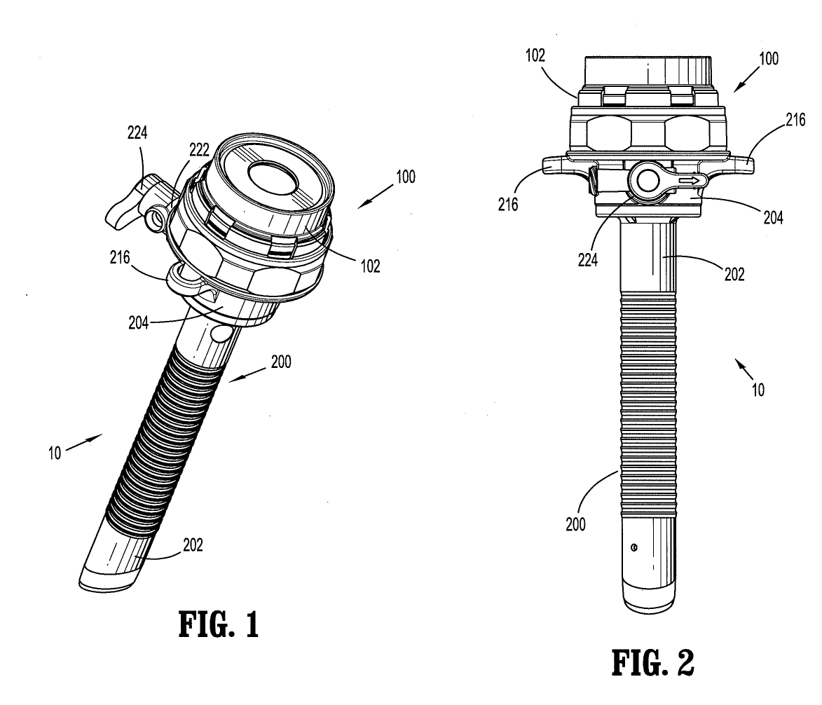

Referring now to the drawings, in which like reference numerals identify identical or substantially similar parts throughout the several views, FIGS. 1-2 illustrate a portal system 10 of the present disclosure incorporating seal assembly 100 mounted to an access device such as cannula assembly 200. Cannula assembly 200 may be any portal member suitable for the intended purpose of accessing a body cavity and typically defines a passageway permitting introduction of instruments therethrough. Cannula assembly 200 is particularly adapted for use in laparoscopic surgery where the peritoneal cavity is insufflated with a suitable gas, e.g., CO₂, to raise the cavity wall from the internal organs therein. Cannula assembly 200 is typically used with an obturator assembly (not shown) which may be blunt, a non-bladed, or a sharp pointed instrument positionable within the passageway of the cannula assembly 200. The obturator assembly is utilized to penetrate the abdominal wall or introduce the cannula assembly 200 through the abdominal wall, and then subsequently is removed from the cannula assembly 200 to permit introduction of the surgical instrumentation utilized to perform the procedure through the passageway.

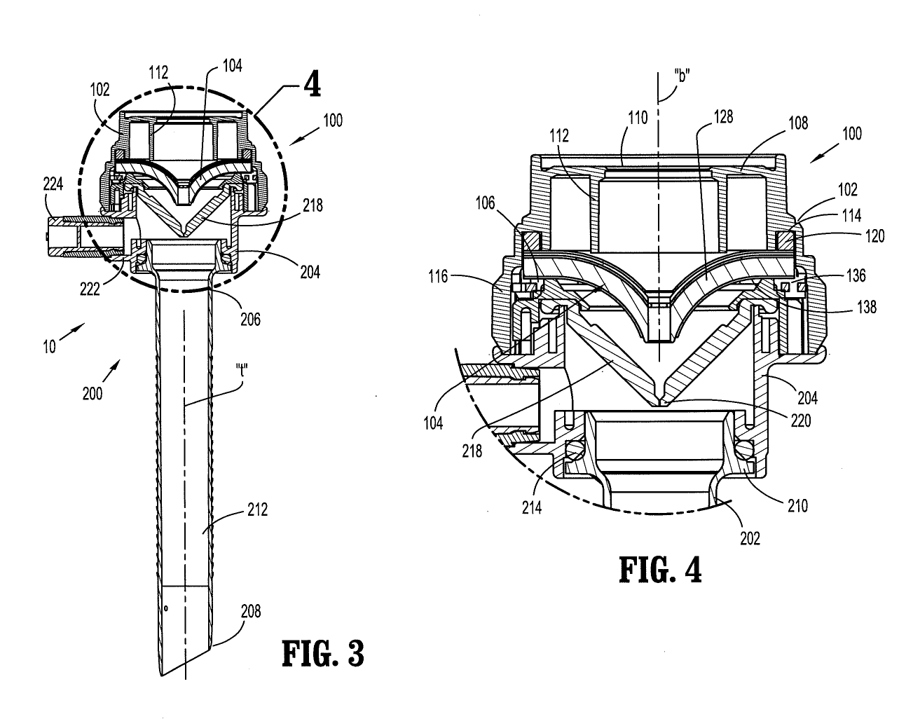

With respect to FIGS. 3-4, in conjunction with FIGS. 1-2, cannula assembly 200 includes cannula sleeve 202 and cannula housing 204 mounted to a proximal end of the cannula sleeve 202. Cannula sleeve 202 defines a longitudinal axis “t” extending along the length of the cannula sleeve 202 and has proximal (or leading) and distal (or trailing) ends 206, 208. Mounted adjacent proximal end 206 of cannula sleeve 202 is sleeve flange 210. Cannula sleeve 202 further defines an internal longitudinal passage 212 dimensioned to permit passage of surgical instrumentation. Cannula sleeve 202 may be formed of any suitable medical grade material, such as stainless steel or other rigid materials, including polymeric materials, such as polycarbonate, or the like. Cannula sleeve 202 may be transparent or opaque. The diameter of cannula sleeve 202 may vary, but, typically ranges from about 3.0 to about 18 mm. Cannula sleeve 202 may or may not include means for facilitating retention of the cannula sleeve 202 within tissue. Such means may include a plurality of locking elements or ribs such as, e.g., the locking arrangement disclosed in commonly assigned U.S. patent application Ser. No. 11/170,824 to Smith filed Jun. 30, 2005, the entire contents of the '824 disclosure being hereby incorporated by reference herein.

Cannula housing 204 may be connected to sleeve flange 210 of cannula sleeve 202 by conventional means including a bayonet coupling, a threaded connection, snap fit, ultrasonic welding or any other means envisioned by one skilled in the art including, e.g., adhesive means. Cannula assembly 200 may also incorporate 0-ring seal 214 disposed between sleeve flange 210 and cannula housing 204 to assist in sealing the interior passages of cannula assembly 200. Cannula housing 204 may be a single monolithically formed unit or composed of several components connected to each other through any of the aforementioned connection means. Cannula housing 204 further includes diametrically opposed housing grips 216 dimensioned and arranged for gripping engagement by the fingers of the clinician. Additionally or alternatively, suture anchors or filaments may extend from cannula housing 210, e.g., from housing grips 216, for attachment to the epidermis of the patient.

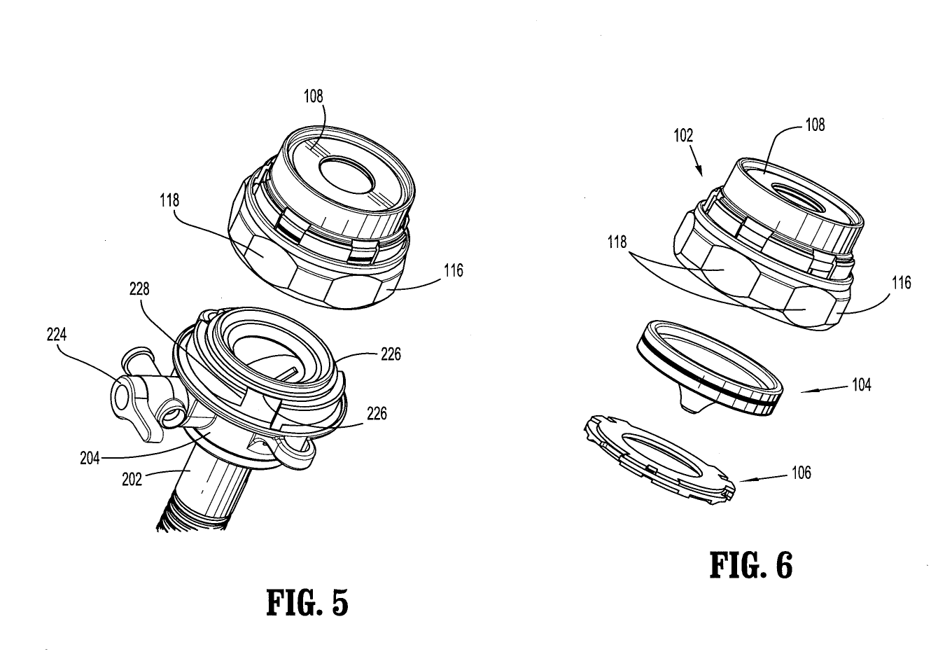

With reference to FIGS. 1-5, cannula housing 204 further includes valve 218. Valve 218 may be a zero-closure valve such as a duck-bill valve having a slit 220 which is adapted to close in the absence of a surgical object and/or in response to insufflation gases of the pressurized cavity. In the alternative, valve 218 may be a gel seal, balloon valve, or a flapper valve. Cannula housing 204 may further include port 222 to which stop cock valve 224 is attached. Port 222 permits the introduction of insufflation gases though cannula sleeve 202 via opening of stop cock valve 224 to assist in maintaining the integrity of the pneumoperitoneum. Stop cock valve 224 may be any conventional valve. As best depicted in FIG. 5, cannula housing 204 further includes at least one, e.g., three, peripheral grooves 226. Grooves 226 extend in an axial direction and are preferably equidistally spaced about the periphery of the cannula housing 204. Grooves 226 assist in releasably mounting seal assembly 100 to cannula assembly 200.

Referring now to FIGS. 5-6, in conjunction with FIGS. 3-4, seal assembly 100 will be discussed in detail. Seal assembly 100 includes seal housing, generally identified as reference numeral 102, composite object seal 104 which is disposed within the seal housing 102 and internal locking element 106. Seal housing 102 houses the sealing components of the assembly and defines the outer valve or seal body of the seal assembly 100. Seal housing 102 defines central seal housing axis “b” which is preferably parallel to the axis “t” of cannula sleeve 202 and, more specifically, coincident with the axis “t” of the cannula sleeve 102 when seal assembly 100 is mounted to cannula assembly 200. Seal housing 102 may be integrally or monolithically formed as a single unit or may incorporate multiple components which, when assembled together, form the seal housing 102. In the illustrated embodiment, seal housing 102 is a single unit formed of a suitable polymeric material. Suitable polymeric materials include polycarbonates polystyrenes, ABS or any other material contemplated by one skilled in the art.

Seal housing 102 includes proximal end wall 108 defining central aperture 110 and internal annular collar 112 depending from the end wall 108 and coaxially arranged about seal housing axis “b”. Central aperture 110 and annular collar 112 receive the surgical object and collectively define an internal dimension or diameter adapted to permit passage of relatively large sized instruments. Annular collar 112 also may limit the degree of lateral or offset movement of the surgical object, e.g., surgical instrument, relative to seal axis “b” by, defining an outer limit of movement of the instrument. Seal housing 102 further defines internal peripheral recess or channel 114 (FIG. 4) approximately adjacent the longitudinal midpoint of the seal housing 102 for receiving a component of object seal 104 as will be discussed.

Seal housing 102 further includes mounting collar 116 adjacent its distal end. Mounting collar 116 may be selectively releasably connectable to cannula housing 204 to cooperatively releasably couple seal assembly 100 to cannula assembly 200. Various means for releasably securing or connecting mounting collar to cannula housing are envisioned including a bayonet coupling, snap-fit, frictional fit, tongue and groove arrangement, threaded arrangement, cam-lock mechanisms or the like. One methodology contemplated will be discussed in greater detail hereinbelow. Alternatively, seal housing 102 may be permanently secured to cannula housing 204. Mounting collar 116 may have an irregular exterior surface to facilitate engagement by the clinician. In one embodiment, mounting collar includes an arrangement of spaced recesses 118 to assist in gripping of seal assembly 100.

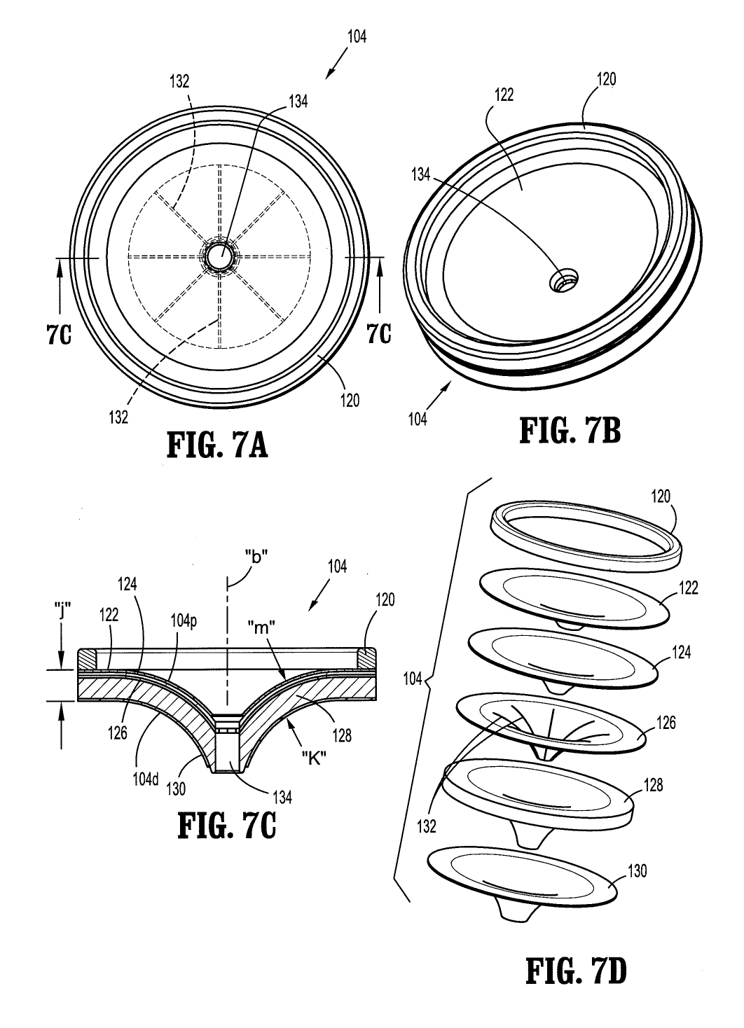

With particular reference to FIGS. 7A-7D, in view of FIGS. 4 and 6, composite object seal 104 will be discussed in detail. Object seal 104 includes from, proximal to distal, retention member 120, first fabric segment 122, elastomeric segment 124, second fabric segment 126, foam segment 128 and third fabric segment 130. Retention member 120 is general annular or ring-like in shape and may be fabricated from a suitable biocompatible relatively rigid material such as polypropylene, nylon, ABS, polycarbonate, stainless steel, titanium or any other suitable material. Retention member 120 assists in mounting object seal 104 within seal housing 102 by its reception within peripheral channel 114 of the seal housing 102 (FIG. 4). First fabric segment 122, second fabric segment 126 and third fabric segment 130 may each be in the form of disc-shaped layers. Fabric segments 122, 126, 130 generally enhance the structural integrity of object seal 104 by providing a support lattice or structure to encapsulate and support foam segment 128. Fabric segment 122,130 also may prevent foam segment 128 of object seal 104 from contact with the instrument during insertion and possibly withdrawal of the instrument through object seal 104. Fabric segments 122, 126, 130 also enhance seal durability and may reduce object or instrument insertion forces. A suitable fabric material for each of fabric segments 122, 126, 130 includes a SPANDEX™ material containing, e.g., 20% LYCRA™ which is commercially available from Milliken of South Carolina. The fabric may comprise a woven, knitted, braided, or non-woven material of polymeric materials. Other fabric materials are also envisioned. For example, a synthetic material such as nylon, Kevlar (Trademark of E.I. DuPont de Nemours and Company) or any other material that will expand and compress about an instrument inserted therethrough is envisioned. In addition, the fabric may be coated, e.g., on its interior with urethane, silicon or other flexible lubricious materials to facilitate passage of an instrument or other object, through the seal.

Each fabric segment 122, 126, 130 may define an aperture, opening or passage to permit passage of the surgical object. Single or multiple intersecting slits within any one or more of fabric segments 122, 126, 130 are also envisioned. For example, second fabric segment 126 may define at least one, possibly, a plurality of slits 132 extending outwardly from seal axis “b”. In one embodiment, slits 132 are substantially linear and extend radially outwardly relative to seal axis “b”. Other arrangements are envisioned including non-linear slits, serpenditious slits, intersecting slits. Slits 132 may be equidistally and radial spaced about seal axis. Slits 132 may assist in reducing insertion and withdrawal forces needed to advance the object into the surgical site by reducing radial constriction of the inner areas of fabric segment 130 about the object.

With continued reference to FIGS. 7A-7D, elastomeric segment 124 in fabricated from a suitable elastomeric or thermoplastic polymer which may exhibit less elasticity than the elasticity of the material of foam segment 128. Elastomeric segment 124 provides protection to foam segment 128 by minimizing the potential of puncture with the surgical object, e.g., the instrument during insertion. Elastomeric segment 128 may be fabricated from a polyisoprene; however, other elastomers are also envisioned including neoprene, silicone, butyl, nitrile and Buna-N.

Foam segment 128 is fabricated from a foam material (closed cell or open cell) such as, in one embodiment, a thermoplastic material comprising a foaming agent. Foam segment 128 may be the primary sealing component about the surgical object. In one embodiment, foam segment 128 is fabricated from a polyisoprene impregnated or injected with a foaming agent including, e.g., CELOGEN™, EXPANDEX™, and OPEX™ chemical foaming agents. Foam segment 128 has sufficient elasticity to bend and deform about the inserted instrument while conforming about the outer dimensioning of the object, e.g., instrument, thereby establishing a fluid tight seal about the object. Foam segment 128 is sufficiently compliant to absorb off axis motion of the instrument. Moreover, the compliant characteristics of foam segment 128 may substantially minimize the formation of a gap around the instrument during off-set manipulation of the instrument. The presence of a gap would otherwise permit the undesired release of gases from the underlying pneumoperitioneum.

In the assembled condition of object seal 104, best depicted in FIG. 7C, the object seal 104 defines a generally tapered or funneled profile whereby the inner area of the object seal 104 slopes at an oblique angle with respect to the seal axis “b”. The funneled characteristic may assist in guiding the instrument toward central aperture 134 during initial introduction of the instrument. The funneled characteristic also may substantially minimize the potential of inversion of object seal 104 during withdrawal of the instrument. More particular, object seal 104 defines proximal seal surface 104p adjacent fabric segment 122 and distal seal surface 104p adjacent fabric segment 130 defining an arcuate, parabolic or hyperbolic profile. In one embodiment, proximal seal surface 104p may define a surface portion having a radius of curvature “m” ranging from about 0.540 inches to about 0.620 inches, and distal seal surface 104d may define a surface portion having a radius of curvature “k” ranging from about 0.430 inches to about 0.500 inches. Other dimensioning and radii of curvatures are also envisioned. The axial length “j” of object seal 104 ranges from about 0.47 inches to about 0.53 inches (excluding retention member 120). This overall dimensioning effected through the sloped arrangement provides an exaggerated funneled profile to object seal 104, which, facilitates directing or funneling of the surgical object through object seal 104 and, also, minimizes the potential of inversion of the object seal 104 during withdrawal of the object.

Object seal 104 may be manufactured via conventional means. In one method, fabric segments 122, 126, 130, elastomeric segment 124 and foam segment 128 may be compression molded while the elastomeric material of the elastomeric segment 124 and/or the foam material of the foam segment 128 is subjected to heat to at least partially embed the fabric material of fabric segments 122, 126, 130 into the elastomeric and/or foam segments 124, 128. Alternatively, the components of object seal 104 may be attached with adhesives, cements, or the like. The methodologies for attaching fabric segments 122, 126, 130 to one or both of elastomeric and foam segments 124, 128 as disclosed in certain embodiments of the U.S. Pat. No. 6,702,787 to Racenet also may be utilized. The entire disclosure of U.S. Pat. No. 6,702,787 to Racenet is hereby incorporated by reference herein. Another method for fabricating seal 104 is disclosed in (H-US-00875), the entire contents of which are hereby incorporated by reference herein. Once object seal 104 is assembled or manufactured, central seal aperture 134 may be punched through the composite materials with a die punch or made via a molding process which provides the seal aperture 134. Alternatively, the respective components of object seal 104 may be provided with cooperative aperture or slits and then assembled via any of the aforementioned methodologies.

In the alternative, object seal 104 may be a substantially flat or planar septum seal having an aperture, slit or the like. Object seal 104 also may comprise a gel composition or very-soft thermoplastic elastomer in addition to, or in lieu of, foam segment 128. Gels and soft thermoplastic materials contemplated for use are known under the trade names VERSAFLEX™, FLEXPLAST™, DYANFLEX™ and MONPRENE™. Other suitable materials include soft silicone and polyurethane composites. These materials may be adapted to be sufficiently pliable.

Object seal 104 may incorporate a lubricant or a therapeutic or pharmacological agent. Suitable lubricants include a coating of hydrocyclosiloxane prepared by plasma polymerization process. Such a coating is disclosed in U.S. Pat. No. 5,463,010 to Hu et al., the entire contents of which is hereby incorporated by reference. Examples of therapeutic or pharmacological agents include antimicrobials, antibacterials, hemostatic, moisture-providing agents, such as saline, healing agents, lubricious agents, analgesics, antiseptics, growth factors, and/or anti-inflammatory agents.

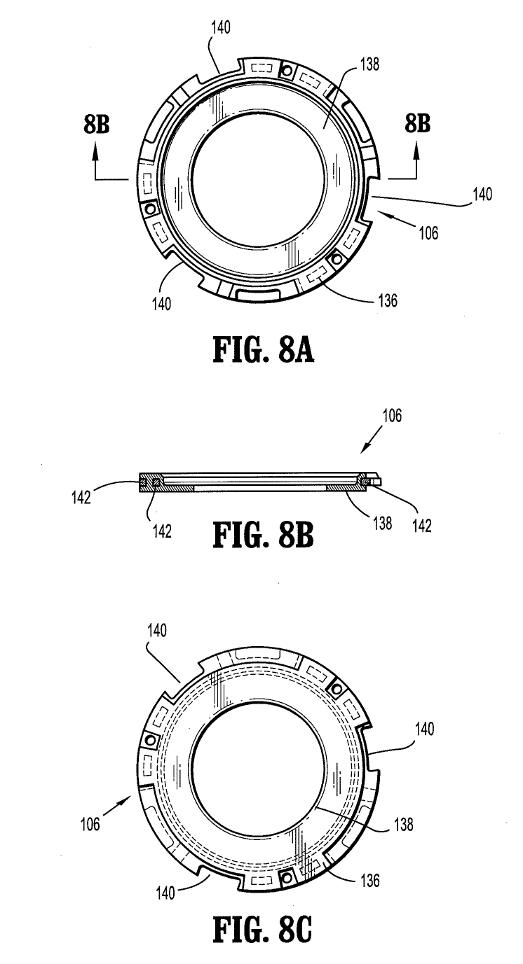

Referring now to FIGS. 8A-8C, in conjunction with FIGS. 4 and 6, locking ring 106 of seal assembly 100 will be discussed. Locking ring 106 serves a dual function of securing object seal 104 within seal housing 102 and providing an internal seal within the passageway of the assembled seal and cannula assemblies 100, 200. Locking ring 106 may be fabricated from a material exhibiting some resiliency including any elastomeric material hereinabove described. Locking ring 106 includes outer segment 136 and internal gasket segment 138. Outer segment 136 defines a plurality (e.g., three) peripheral recesses 140 equidistally spaced about the periphery. Recesses 140 assist in mounting locking ring 106 within seal housing 102. Internal gasket segment 138 of locking ring 106 may contact distal seal surface 104d of object seal 104 in the assembled condition of seal assembly 100. Outer segment 136 and internal gasket segment 138 may be monolithically formed or may consist of separate components received to each other by conventional means. Internal gasket segment 138 may be more elastic than outer segment 136. Outer segment 136 may incorporate first and second O-ring seals 142 within annular recesses of the outer segment to assist in sealing the internal opening of seal housing 102.

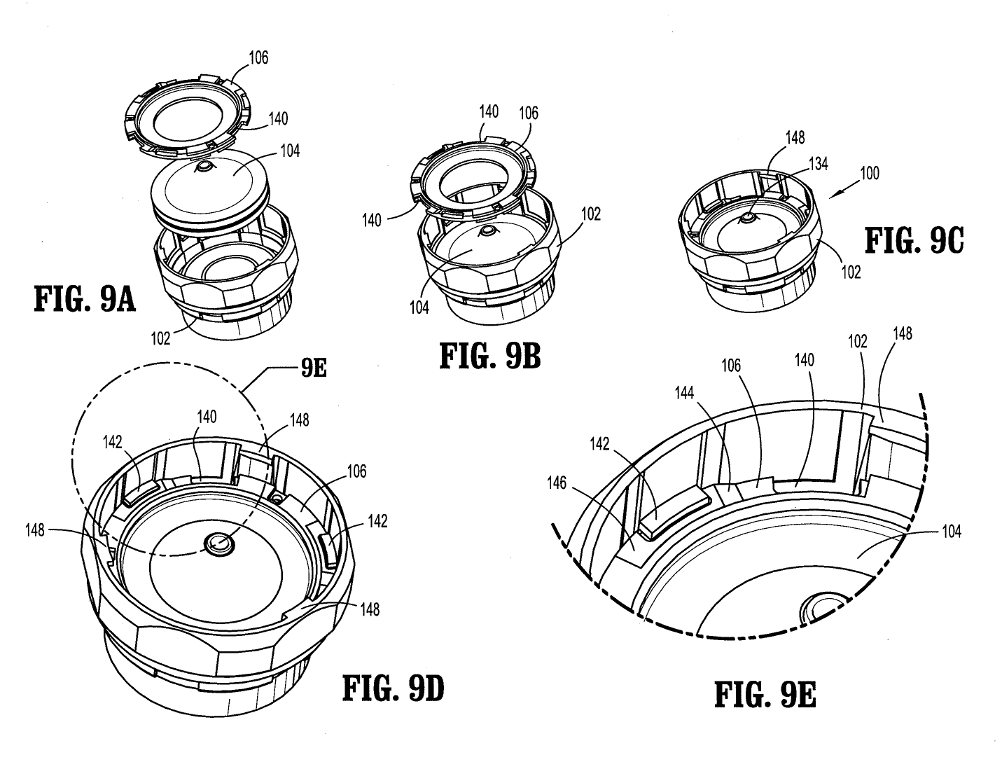

The assembly of seal assembly 100 will now be discussed. With reference to FIGS. 9A-9F, in conjunction with FIG. 4, object seal 104 is aligned with the internal area of seal housing 102 and advanced within the seal housing 102 whereby retention member 120 is received within channel 114 of seal housing 102 (see also FIG. 4). Locking ring 106 is thereafter positioned whereby peripheral recesses 140 of the locking ring 106 are aligned with mounting tabs 142 extending radially inwardly from the interior wall of seal housing 102. In particular, as best depicted in FIGS. 9D-9E, mounting tabs 142 are disposed within the interior of seal housing 102. At least two, e.g., three, mounting tabs 142 and associated peripheral recesses 140 of locking ring 106 are envisioned. Locking ring 106 is advanced into seal housing 102 with mounting tabs 142 being received within peripheral recesses 140 of the locking ring 106. Thereafter, locking ring 106 is rotated relative to seal housing 102 to cause mounting tabs 142 of seal housing 102 to ride along peripheral surfaces on the underside of the locking ring 106 to be out of alignment with respective peripheral recesses 140. In this arrangement as depicted in FIG. 9E, mounting tabs 140 are secured against the underside of locking ring 106. It is envisioned that mounting tabs 140 may initially ride along inclined or cam surfaces 144 provided on the underside of locking ring 106 and adjacent recesses 140 to draw the locking ring 106 against object seal 104. It is further envisioned that the underside of locking ring 106 may have locking recesses 146 formed therein to assist in securing locking tabs 106 relative to seal housing 102. FIG. 9D illustrates the assembled seal assembly 100.

Seal assembly 100 may be associated with, or joined to, cannula assembly 200 in a variety of ways. In a preferred embodiment, seal housing 102 of seal assembly 100 and cannula housing 204 of cannula assembly 200 are adapted to detachably engage each other, e.g., through a bayonet lock, threaded attachment, latching attachment, or like mechanical means. In one preferred arrangement, seal housing 102 includes at least two, preferably, three housing locking detents 148 as best depicted in FIGS. 9D-9E. Locking detents 148 are aligned with and subsequently received within, axial recesses of cannula housing 204 (FIG. 5) during mounting of seal assembly 100 to cannula assembly 200. In one embodiment, seal housing 102 and cannula housing 104 are rotated relative to each other to position housing locking detents beneath on annular ledge 228 defined within the cannula housing 104 to receive the components. Alternatively, seal housing 102 and cannula housing 104 may be releasably secured to each other via a friction fit or the like. Seal assembly may be mounted to cannula assembly 100 before, during, or after, application of cannula assembly 200 within the operative site.

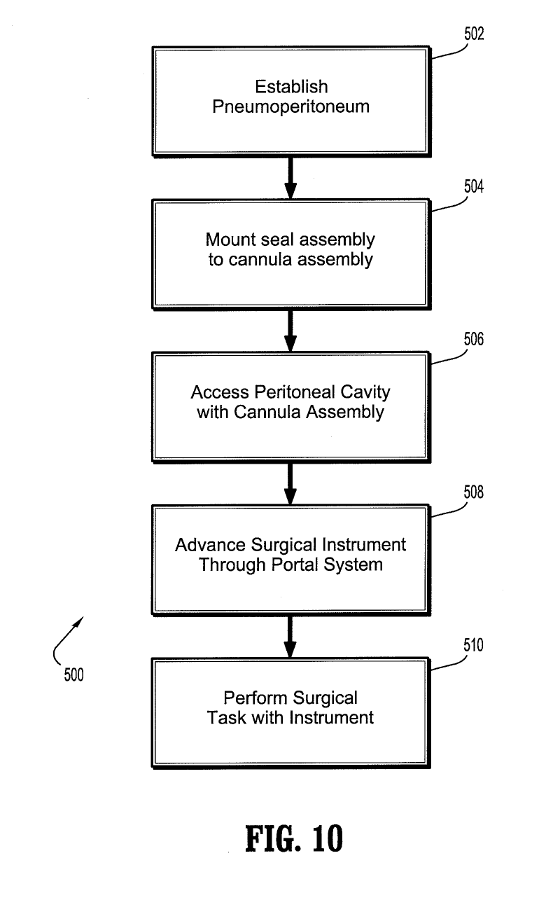

FIG. 10 is a flow chart 500 illustrating the use of seal assembly 100 and cannula assembly 200 in connection with the performance of a surgical task during a laparoscopic procedure. The peritoneal cavity is insufflated to establish the pneumoperitonum (Step 502). Seal assembly 100 is mounted to cannula assembly 200 (step 504) as discussed hereinabove. The assembled portal system 10 is introduced into an insufflated abdominal cavity typically utilizing a sharp or non-blade trocar obturator to access the cavity (step 506) and the obturator is removed. An instrument may be advanced through portal system 10 (step 508) by inserting the instrument into seal assembly 100 through object seal 104 whereby the portions defining aperture 134 of the object seal 104 stretch to accommodate the instrument in substantial sealed relation therewith. The instrument is distally passed through valve 218 and into the body cavity. The desired surgical task is performed with the instrument (510). During manipulation of the instrument, at least the foam material of foam segment 128 conforms about the instrument to prevent formation of any gaps on opposed sides of the instrument. Fabric segments 122, 126, 130, and elastomeric segment 124 assist in supporting and preserving the integrity of foam segment 128 during insertion, manipulation and withdrawal of the surgical instrument.

It will be understood that various modifications and changes in form and detail may be made to the embodiments of the present disclosure without departing from the spirit and scope of the invention. Therefore, the above description should not be construed as limiting the invention but merely as exemplifications of preferred embodiments thereof. Those skilled in the art will envision other modifications within the scope and spirit of the present invention as defined by the claims appended hereto. Having thus described the invention with the details and particularity required by the patent laws, what is claimed and desired protected is set forth in the appended claims.

What is claimed is:

1. A surgical portal assembly, which comprises:
a portal adapted to provide access to underlying tissue and having a longitudinal opening extending along a longitudinal axis of the portal, and defining proximal and distal ends; and
a seal including internal surfaces having a passage for reception and passage of a surgical object in substantial sealed relation therewith and defining a seal axis, the seal including a foam segment comprising a foam material, and a fabric segment comprising a fabric material and being mounted relative to the foam segment.

2. The surgical portal assembly according to claim 1 wherein the fabric segment is disposed adjacent one of the proximal and distal surfaces of the foam segment.

3. The surgical portal assembly according to claim 2 wherein the fabric segment includes a fabric layer, the fabric layer in juxtaposed relation with the one of the proximal and distal surfaces of the foam segment.

4. The surgical portal assembly according to claim 3 wherein the fabric layer includes slots therethrough to facilitate passage of the surgical object through the seal.

5. The surgical portal assembly according to claim 4 wherein the slots are arranged to extend radially outwardly relative to the seal axis.

6. The surgical portal assembly according to claim 3 including first and second fabric layers mounted in juxtaposed relation with respective proximal and distal surfaces of the foam segment.

7. The surgical portal assembly according to claim 3 wherein the seal further includes an elastomeric segment comprising an elastomeric material having less elasticity than the foam material of the foam segment.

8. The surgical portal assembly according to claim 7 wherein the elastomeric segment is mounted in juxtaposed relation to the fabric layer.

9. The surgical portal assembly according to claim 8 wherein the seal includes, from proximal to distal, the elastomeric segment, the fabric layer and the foam segment.

10. The surgical portal assembly according to claim 9 further including a second layer of fabric mounted adjacent the distal surface of the foam segment.

11. The surgical portal assembly according to claim 10 further including a third layer of fabric mounted adjacent a proximal surface of the elastomeric segment.

12. The surgical portal assembly according to claim 3 wherein the seal includes an outer seal area and an inner seal area, the inner seal area generally tapering in a distal direction to define a general funnel configuration.

13. The surgical portal assembly according to claim 3 wherein the seal includes an outer seal area and an inner seal area, the inner seal area defining a general sloped portion to facilitate insertion of the surgical object and minimize potential of inversion of the inner seal area upon withdrawal of the surgical object.

14. The surgical portal assembly according to claim 2 wherein the portal includes a portal housing and a portal sleeve extending from the portal housing.

15. A surgical portal assembly according to claim 14 wherein the seal includes a substantially annular retention member, the retention member being received within a corresponding annular recess of the portal housing to assist in mounting the seal within the portal housing.
